(19) United States
(12) Patent Application Publication
(10) Pub. No.: US 2023/0035679 A1
(43) Pub. Date: Feb. 2, 2023

(54) HAIR CARE APPLIANCE

(71) Applicant: LG ELECTRONICS INC., Seoul (KR)

(72) Inventors: Hyunchul KIM, Seoul (KR); Kyoungtae KIM, Seoul (KR); Yunhee KU, Seoul (KR); Rayoung PARK, Seoul (KR); Dongwon KIM, Seoul (KR)

(73) Assignee: LG ELECTRONICS INC., Seoul (KR)

(21) Appl. No.: 17/788,220

(22) PCT Filed: Dec. 31, 2019
(86) PCT No.: PCT/KR2019/018785

§ 371 Date: Jun. 22, 2022

(30) Foreign Application Priority Data
Dec. 23, 2019 (KR) 10-2019-0173383

(51) Int. Cl. A61N 1/04; A45D 2/00; A61N 1/32

(52) U.S. Cl. CPC A61N 1/044; A45D 2/001; A61N 1/325; A45D 19/0041

(57) ABSTRACT

Discussed is a hair care appliance including: a first care body having a first care electrode; a second care body having a second care electrode; and a bending unit connected between the first care body and the second care body. The first care body includes a main substrate electrically connected to the first care electrode, and a processor provided at the main substrate, and the second care body includes a battery electrically connected to the second care electrode, and the bending unit includes an elastic housing and a flexible substrate which is accommodated in the elastic housing and which electrically connects the battery to the main substrate.

### TECHNICAL FIELD

The present disclosure relates to a hair care appliance.

### BACKGROUND ART

Modern people recognize their appearance as an important factor to inform and express their appearance to others, and accordingly, they spend a lot of money and time in appearance management to overcome their flaws in appearance.

Such appearance management may include not only skin care such as the face or weight management but also hair care.

Hair may be damaged due to stress, pollution, nutritional imbalance, etc. or hair loss may occur. In particular, people spend a lot of money in hair styling such as dyeing or perm to make their appearance stand out more. However, in the case of the above-described hair styling, most of methods of applying chemical substances to the hair may cause problems such as damage to the hair or degradation of nutrition.

Accordingly, recently, various devices for hair or scalp management have been released, but most of the devices provide only a rather limited function, such as irradiating light to the scalp.

In addition, although various nutritional products applied to the hair have been released, there is a limit in that nutritional components of these products do not penetrate into the hair smoothly, and thus an effect thereof is halved.

### DISCLOSURE

### Technical Problem

An object of an embodiment of the present disclosure is to provide a hair care appliance for effective penetrating various ingredients for hair health into the hair.

Another object of an embodiment of the present disclosure is to provide a hair care appliance for transmitting and receiving an electrical signal or power through a bending part between care bodies.

Another object of an embodiment of the present disclosure is to provide a hair care appliance having current characteristics for maximizing a care effect.

### Technical Solution

According to an embodiment of the present disclosure, a hair care appliance includes a first care body having a first care electrode formed thereon, a second care body having a second care electrode formed thereon, and a bending part connected between the first care body and the second care body.

The first care body may include a main board electrically connected to the first care electrode, and a processor included in the main board.

The second care body may include a battery electrically connected to the second care electrode.

The bending part may include an elastic housing, and a flexible PCB (FPCB) accommodated in the elastic housing and electrically connecting the battery and the main board.

In some embodiments, the bending part may elastically support the first care body and the second care body to form a predetermined angle.

In some embodiments, the first care electrode and the second care electrode may be formed to face each other.

In some embodiments, the first care electrode may be formed adjacent to one end of the first care body, which is not connected to the bending part, among both ends of the first care body and the second care electrode may be formed adjacent to one end of the second care body, which is not connected to the bending part, among both ends of the second care body.

The first care electrode and the second care electrode may form different polarities

In some embodiments, the hair care appliance may further include a grip electrode formed on one of the first care body and the second care body and electrically connected to the battery. In this case, the first care electrode and the second care electrode may form the same polarity, and the grip electrode may form a different polarity from the first care electrode and the second care electrode.

The grip electrode may be formed on a surface of the one of the care bodies, which does not face another care body, among surfaces of the care bodies.

The grip electrode may be formed adjacent to one end of the one of the care bodies, which is connected to the bending part, among both ends of the one of the care bodies.

In some embodiments, the processor may perform control to alternately form a (+) polarity and a (−) polarity in each of the first care electrode and the second care electrode for a preset time from a time at which the first care electrode and the second care electrode come in contact with hair during driving of the hair care appliance.

### Advantageous Effects

According to an embodiment, a hair care appliance may maximize a hair care effect by effectively penetrating a nutrient component applied to the hair into the hair through an iontophoresis scheme.

A main board including a main component such as a processor and a battery for supplying power may be provided in different care bodies, and thus a problem such as increase in volume or imbalance in weight due to concentration of components on a specific care body may be overcome.

A flexible PCB may be provided in a bending part connected between care bodies, and thus electrical connection between a battery and a main board may be maintained.

When a grip electrode is formed in a hair care appliance, the grip electrode may be disposed adjacent to one end of a care body, which is connected to the bending part, among both ends of the care body. Thus, when the hair care appliance is used, a user's hand and the grip electrode may smoothly come into contact, thereby improving user convenience.

The hair care appliance may be driven according to current characteristics for maximizing penetration of a nutrient component into the hair, thereby maximizing an absorption rate of the nutrient component compared with power consumption.

### DESCRIPTION OF DRAWINGS

FIG. 1 is a perspective view of a hair care appliance according to an embodiment of the present disclosure.

FIG. 2 is a longitudinal cross-sectional view for explaining the internal configuration of a hair care appliance according to an embodiment of the present disclosure.

FIG. 3 is a diagram showing a position change of care bodies when a hair care appliance is used.

FIGS. 4 to 5 are diagrams for explaining a hinge part provided on a hair care appliance according to another embodiment of the present disclosure.

FIG. 6 is a perspective view of a hair care appliance according to another embodiment of the present disclosure.

FIG. 7 is a schematic block diagram showing control components of a hair care appliance according to an embodiment of the present disclosure.

FIGS. 8 to 9 are exemplary graphs related to the characteristics of current applied to care electrodes of a hair care appliance.

FIG. 10 is a diagram showing the case in which a hair care appliance is used for the hair according to an embodiment of the present disclosure.

FIGS. 11 to 13 are diagrams for explaining an operation principle of a hair care appliance according to an embodiment of the present disclosure.

### BEST MODE

Description will now be given in detail according to exemplary embodiments disclosed herein, with reference to the accompanying drawings. For the sake of brief description with reference to the drawings, the same or equivalent components may be provided with the same reference numbers, and description thereof will not be repeated. In general, a suffix such as “module” and “unit” may be used to refer to elements or components. Use of such a suffix herein is merely intended to facilitate description of the specification, and the suffix itself is not intended to give any special meaning or function. In the present disclosure, that which is well-known to one of ordinary skill in the relevant art has generally been omitted for the sake of brevity. The accompanying drawings are used to help easily understand various technical features and it should be understood that the embodiments presented herein are not limited by the accompanying drawings. As such, the present disclosure should be construed to extend to any alterations, equivalents and substitutes in addition to those which are particularly set out in the accompanying drawings.

It will be understood that although the terms first, second, etc. may be used herein to describe various elements, these elements should not be limited by these terms. These terms are generally only used to distinguish one element from another.

It will be understood that when an element is referred to as being “connected with” another element, the element can be directly connected with the other element or intervening elements may also be present. In contrast, when an element is referred to as being “directly connected with” another element, there are no intervening elements present.

A singular representation may include a plural representation unless it represents a definitely different meaning from the context.

Terms such as “include” or “has” are used herein and should be understood that they are intended to indicate an existence of several components, functions or steps, disclosed in the specification, and it is also understood that greater or fewer components, functions, or steps may likewise be utilized.

Hereinafter, embodiments of the present disclosure will be described in detail with reference to the accompanying drawings.

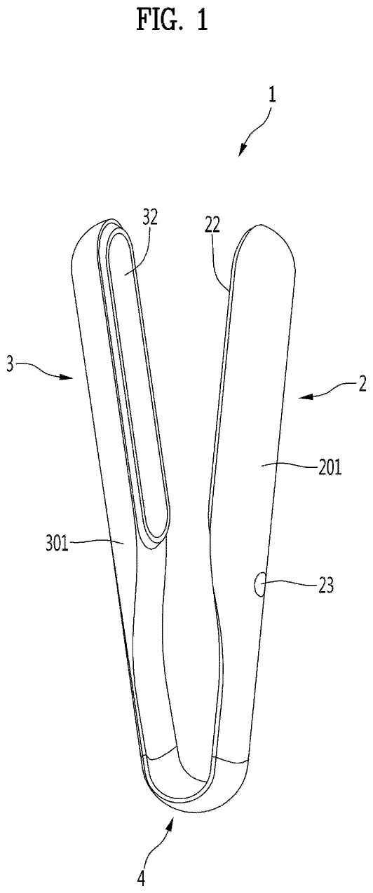

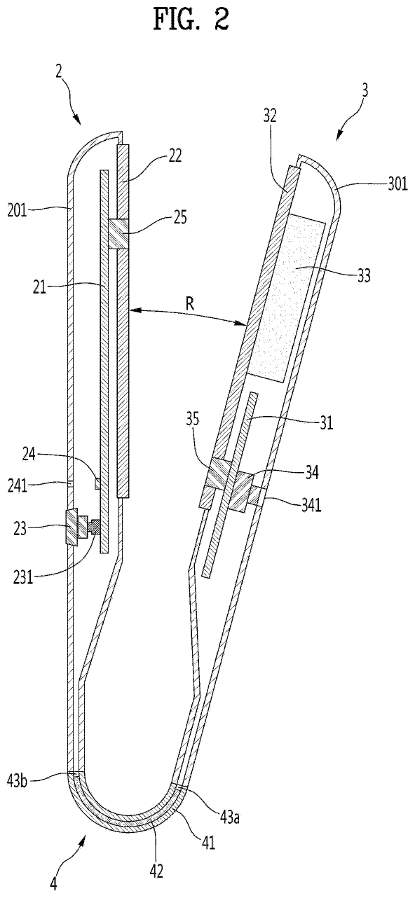

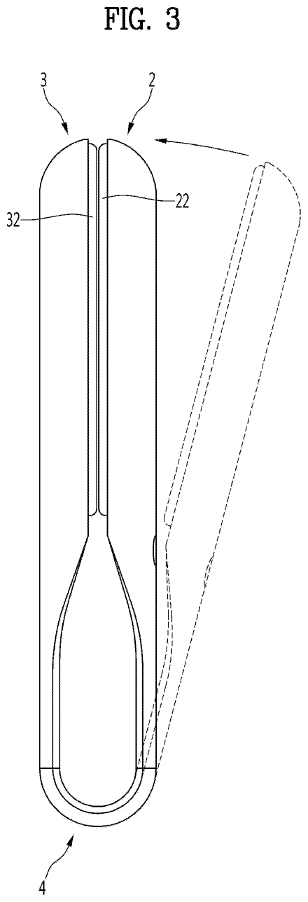

FIG. 1 is a perspective view of a hair care appliance according to an embodiment of the present disclosure. FIG. 2 is a longitudinal cross-sectional view for explaining the internal configuration of a hair care appliance according to an embodiment of the present disclosure. FIG. 3 is a diagram showing a position change of care bodies when a hair care appliance is used.

Referring to FIGS. 1 to 3, a hair care appliance 1 according to an embodiment of the present disclosure may be a device for more effectively absorbing or penetrating nutrients into the hair based on iontophoresis technology.

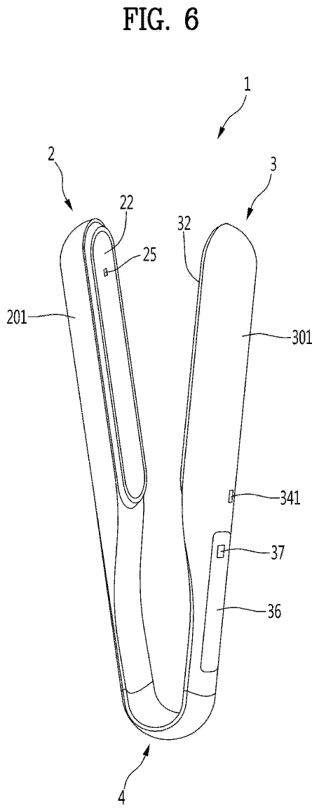

In detail, the user may drive the hair care appliance 1 after applying a nutrient containing a nutrient component (e.g., protein such as keratin or collagen or amino acid) to the hair. The hair care appliance 1 may apply current to form different polarities in the two care electrodes 22 and 23 or the same (−) electrode (refer to the embodiment of FIG. 6). The user may hold the hair care appliance 1 and may press the hair between the care electrodes 22 and 32 to contact the hair. The nutrient component may form a (−) polarity when mixed with water or the like, and accordingly, as current is applied, the nutrient component with a (−) polarity present on a surface of the hair positioned between care electrodes 22 and 32 may penetrate into the hair (cortex or medulla) by electrical repulsion and/or attraction.

The hair care appliance 1 may include a first care body 2, a second care body 3, and a bending part 4 connected between the first care body 2 and the second care body 3.

The hair care appliance 1 may have an overall ‘U’ or ‘V’ shape. When separate external force is not applied to the hair care appliance 1, the bending part 4 may elastically support the first care body 2 and the second care body 3 to form a predetermined angle R (refer to FIG. 2) with each other. For example, the predetermined angle R may be implemented as an acute angle, thereby improving ease of storage of the hair care appliance 1.

The bending part 4 may be formed of a material having elasticity (ABS or poly oxy methylene (POM)) and may be deformed by external force applied from the user when the hair care appliance 1 is used. As shown in FIG. 3, as the bending part 4 is deformed, the first care body 2 and the second care body 3 may be positioned side by side. When the external force is released, the first care body 2 and the second care body 3 may be positioned to form a predetermined angle again by elastic restoring force of the bending part 4.

Respective cases 201 and 301 of the first care body 2 and the second care body 3 may generally have a bar shape. The first care body 2 and the second care body 3 may have a shape symmetrical to each other, but the present disclosure is not limited thereto.

The first care body 2 may include a first care electrode 22, and the second care body 3 may include a second care electrode 32. As shown in FIG. 1, the first care electrode 22 and the second care electrode 32 may form different polarities.

The first care electrode 22 and the second care electrode 32 may be formed to have a predetermined length along a longitudinal direction of the cases 201 and 301. Each of the first care electrode 22 and the second care electrode 32 may be formed closer to one end that is not connected to the bending part 4 among both ends of the cases 201 and 301, but the present disclosure is not limited thereto.

The first care electrode 22 and the second care electrode 32 may be provided on opposite surfaces of the cases 201 and 301. Accordingly, when the hair care appliance 1 is used, the first care electrode 22 and the second care electrode 32 may be in close contact with each other, and the hair care appliance 1 may be in contact with the hair positioned between the first care electrode 22 and the second care electrode 32.

As the first care electrode 22 and the second care electrode 32 have different polarities, mutual attraction may be applied to each other. Accordingly, the user may maintain the close contact state between the care electrodes 22 and 32 with little force, and thus the hair care appliance 1 may be used more conveniently.

The first care electrode 22 and the second care electrode 32 may be formed of electrically conductive metal such as stainless steel (SUS).

Any one (e.g., the first care body 2) of the first care body 2 and the second care body 3 may include at least one input interface (e.g., at least one button 23) for setting on and off or an operation mode of the hair care appliance 1.

Referring to FIG. 2, the first care body 2 may include a main board 21, the first care electrode 22, and the input interface (e.g., at least one button 23), and the second care body 3 may include a sub board 31, the second care electrode 32, a battery 33, and a speaker 34. For convenience of description, components such as a frame or a wire inside the first care body 2 and the second care body 3 are not illustrated.

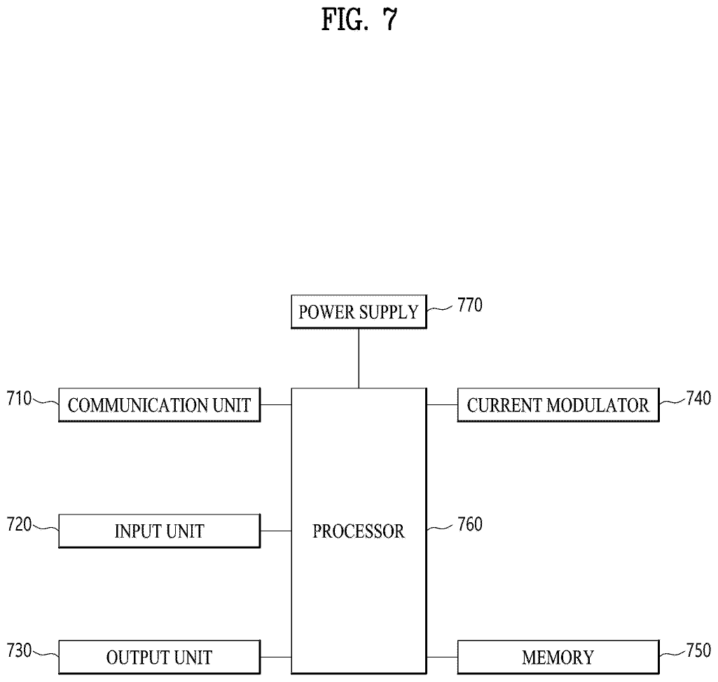

The main board 21 may include main control components such as a processor, a memory, and a communication unit. The control components will be described in more detail below with reference to FIG. 7.

The first care electrode 22 may be electrically connected to the main board 21. For example, an opening for exposing one surface of the first care electrode 22 to the outside may be formed in a first case 201, and the first care electrode 22 may be mounted on the first case 201 through the opening.

The main board 21 may include at least one connector 231 connected to the at least one button 23. A processor 760 (refer to FIG. 7) may detect manipulation of the button 23 by acquiring a signal from the connector 231 corresponding to a manipulated button when one of the at least one button 23 is manipulated.

In some embodiments, a light source 24 for informing a user of information on the state (power or an operation mode) of the hair care appliance 1 may be provided on the main board 21. The light source 24 may be disposed toward one surface (e.g., an outside surface) of the first case 201. A hole 241 for emitting light emitted from the light source 24 to the outside may be formed at a position of the one surface of the first case 201, which corresponds to the light source 24. The light source 24 may be implemented as an LED but is not limited thereto.

The processor 760 may visually inform the user of the state of the hair care appliance 1 by differently controlling the color, brightness, blinking pattern, etc. of the light source 24 according to the state of the hair care appliance 1.

A surface of the main board 21 to which the input interface (the button 23) and the light source 24 are connected and a surface of the main board 21 to which the first care electrode 22 is connected may be different from each other. That is, the main board 21 may be implemented as a double-side board.

The sub board 31 may include a circuit for performing a management operation such as charging of the battery 33.

The second care electrode 32 may be electrically connected to the sub board 31. An opening for exposing one surface of the second care electrode 32 to the outside may be formed in a second case 301, and the second care electrode 32 may be mounted on the second case 301 through the opening.

Although not shown, a light transmitting part (not shown) may be formed at an edge of each of the first care electrode 22 and the second care electrode 32, and at least one light source (e.g., an LED) may be provided in a region of an internal region of the care bodies 2 and 3, which corresponds to the light transmitting part. When current is applied to the first care electrode 22 and the second care electrode 32, the processor 760 may visually inform the user that current is applied by emitting light through the at least one light source.

The battery 33 may supply power to each component of the hair care appliance 1 through the sub board 31.

In some embodiments, the sub board 31 may further include a speaker module 34. The processor 760 may output information on the state (power or an operation mode) of the hair care appliance 1 in the form of voice or sound through the speaker module 34. The speaker module 34 may be disposed toward one surface (e.g., an outside surface) of the second case 301. In this case, a speaker hole 341 for providing voice or sound output from the speaker module 34 to the outside may be formed at a position of the one surface of the second case 301, which corresponds to the speaker module 34. However, in some embodiments, the speaker module 34 may also be included in the main board 21.

As the battery 33 is provided in the second care body 3, there may be a need for a device for electrically connection between the main board 21 and the battery 33 that are provided in the first care body 2.

In particular, the device for electrically connection may be required in the bending part 4 between the first care body 2 and the second care body 3.

In this regard, the bending part 4 is now described, and the bending part 4 may include an elastic housing 41 forming an outer appearance, and a flexible PCB (FPCB) 42 accommodated in the elastic housing 41.

The elastic housing 41 may be formed of an elastic material such as ABS and may be elastically deformed by external force applied from the user. The elastic housing 41 may be integrally implemented, and thus may effectively prevent moisture or dust from penetrating into the FPCB 42 from the outside.

The FPCB 42 may be formed of polyimide (PI), polyester (PET), or glass epoxy (GE). The FPCB 42 may be deformed by the external force with the elastic housing 41 and may be restored to a previous state by elastic restoring force of the elastic housing 41 when the external force is released.

A circuit for electrical connection between the battery 33 and the main board 21 may be formed on the FPCB 42. Electrical connection terminals 43a and 43b may be provided at both ends of the FPCB 42, respectively.

Accordingly, when the first care body 2, the second care body 3, and the bending part 4 are coupled, the battery 33 (or the sub board 31) and the FPCB 42 may be electrically connected through a first electrical connection terminal 43a, and the FPCB 42 and the main board 21 may be electrically connected through a second electrical connection terminal 43b.

The battery 33 and the main board 21 may be electrically connected by the FPCB 42, and components provided in or connected to the main board 21 may be driven using power provided from the battery 33. Since the FPCB 42 is more stable than a wire, it may be possible to minimize a problem such as disconnection of electrical connection between the battery 33 and the main board 21 when the hair care appliance 1 is used for a long time.

That is, according to an embodiment of the present disclosure, the main board 21 including a main control component such as a processor and the battery 33 for providing power may be provided in different care bodies, and thus it may be possible to prevent increase in volume or imbalance in weight due to concentration of components on a specific care body.

The FPCB 42 may be provided in the bending part 4 connected between the care bodies 2 and 3, and thus the battery 33 and the main board 21 may be stably electrically connected to each other.

The first care electrode 22 and the second care electrode 32 may be electrically connected to the battery 33 and may form different polarities, and thus the first care electrode 22 and the second care electrode 32 may function as a terminal for receiving power for charging the battery 33.

For example, a cradle (not shown) functioning as a holder and a charging stand when the hair care appliance 1 is not used may be provided. When the hair care appliance 1 may be mounted on the cradle, each of the first care electrode 22 and the second care electrode 32 may be electrically connected to a power supply formed on the cradle.

As the first care electrode 22 and the second care electrode 32 are connected to the power supply, power may be supplied to the battery 33 from the power supply through the first care electrode 22 and the second care electrode 32.

In some embodiments, the hair care appliance 1 may further include separate power supply terminals 25 and 35. For example, a first power supply terminal 25 may be formed through a partial region of the first care electrode 22 from the main board 21, and a second power supply terminal 35 may be formed through a partial region of the second care electrode 32 from the sub board 31, but the present disclosure is not limited thereto.

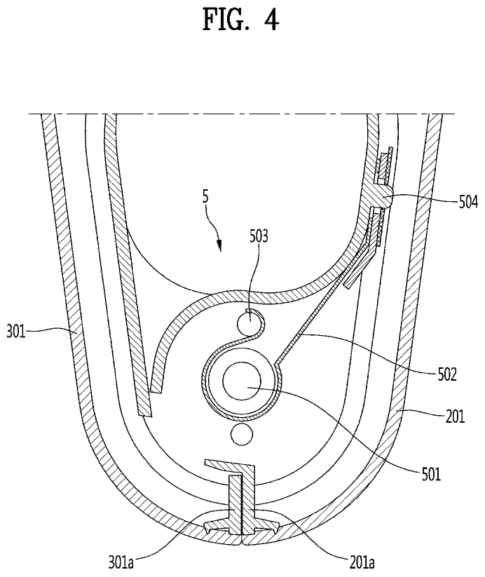

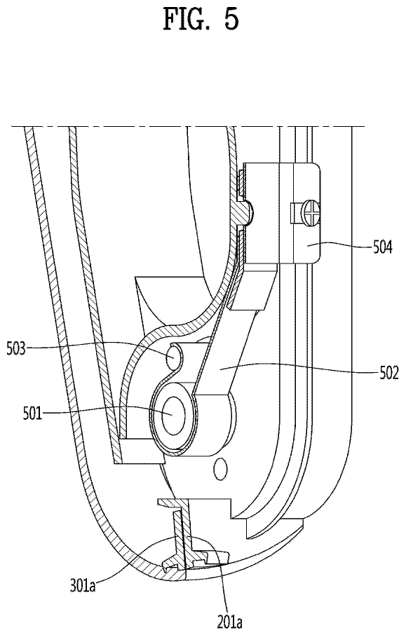

FIGS. 4 to 5 are diagrams for explaining a hinge part provided on a hair care appliance according to another embodiment of the present disclosure.

Referring to FIGS. 4 to 5, in some embodiments, the hair care appliance 1 may include a hinge part 5 instead of the bending part 4 between the first care body 2 and the second care body 3.

When the hinge part 5 is provided, the first case 201 of the first care body 2 and the second case 301 of the second care body 3 may be rotatably connected to each other through the hinge part 5.

In detail, the hinge part 5 may include a hinge axis 501, an elastic body 502 for surrounding at least a portion of the hinge axis 501, and elastic body fixers 503 and 504 for elastically fixing both ends of the elastic body 502, respectively.

The first case 201 and the second case 301 may each be rotatably fixed by the hinge axis 501. In this case, differently from the embodiments of FIGS. 1 to 3, a portion of the first case 201 and a portion of the second case 301 may be in contact with each other.

The elastic body 502 may be formed to surround at least one a portion of an external surface of the hinge axis 501. For example, the elastic body 502 may be implemented as a leaf spring but is not limited thereto.

Both ends of the elastic body 502 may be elastically fixed by the elastic body fixers 503 and 504. A first elastic body fixer 503 and a second elastic body fixer 504 may be fixed to different cases 201 and 301, respectively. For example, the first elastic body fixer 503 may be fixed to the second case 301, and the second elastic body fixer 504 may be fixed to the first case 201.

In this case, as external force is applied to the hair care appliance 1, the first case 201 or the second case 301 may be rotated with respect to the hinge axis 501. Through rotation, the elastic body 502 connected between the first elastic body fixer 503 and the second elastic body fixer 504 may be stretched.

Then, when the external force is released, the stretched elastic body 502 may be contracted by elastic restoring force, and as the elastic body 502 is contracted, the first case 201 or the second case 301 may be rotated to an initial position.

Holders 201a and 301a may be formed on the first case 201 and the second case 301, respectively. The holders 201a and 301a may be in contact with each other when the elastic body 502 is elastically restored, and thus a maximum rotation range of the cases 201 and 301 due to contraction of the elastic body 502 may be limited, and the initial position of the first case 201 or the second case 301 may be maintained.

FIG. 6 is a perspective view of a hair care appliance according to another embodiment of the present disclosure.

Compared with the hair care appliance of FIG. 1, the hair care appliance shown in FIG. 6 may further include a grip electrode 36 formed on surface of any one case (e.g., the second case 301). For example, the grip electrode 36 may be formed on an outside surface of a care body (case), that is, an opposite surface to the surface on which the second care electrode 32 is formed. In other words, the grip electrode 36 may be formed on a surface that is not directed toward the first care body 2 among surfaces of the second care body 3.

As the grip electrode 36 is formed on the outside surface of the case, the user's hand and the grip electrode 36 may come into contact smoothly when the user holds the care bodies 2 and 3 with the hand.

The grip electrode 36 may be disposed closer to one end connected to the bending part 4 among both ends of the second care body 3. Accordingly, when the hair care appliance 1 is used, the user's hand and the grip electrode 36 may come into contact smoothly.

The grip electrode 36 may be formed of an electrically conductive metal such as stainless steel (SUS) or aluminum similar to the care electrodes 22 and 32.

Although not shown, the grip electrode 36 may be electrically connected to the battery 33. As the grip electrode 36 comes into contact with the user's hand and the care electrodes 22 and 32 come into contact with the hair, a closed circuit (closed circuit) for connecting the battery 33, the grip electrode 36, the user's body, the care electrodes 22 and 32, the main board 21, the FPCB 42, and the battery 33 may be configured.

In the closed circuit, the care electrodes 22 and 32 and the grip electrode 36 may form different polarities. For example, the care electrodes 22 and 32 may form the same (−) polarity, and the grip electrode 36 may form a (+) polarity. As the care electrodes 22 and 32 forms a (−) polarity, nutrient components of a (−) polarity present on a surface of the hair may penetrate into the hair by electrical repulsion.

According to the embodiment of FIG. 6, when the grip electrode 36 is provided, at least one of the care electrodes 22 and 32 and the grip electrode 36 may function as a terminal for receiving power for charging of the battery 33. That is, when the hair care appliance 1 is mounted on a cradle (not shown), at least one of the care electrodes 22 and 32 and each grip electrode 36 may be electrically connected to a power supply provided in the cradle.

As at least one of the care electrodes 22 and 32 and each grip electrode 36 are connected to the power supply, power may be supplied to the battery 33 from the power supply through the care electrodes 22 and 32 and the grip electrode 36. In some embodiments, the hair care appliance 1 may further include a power supply terminal 37 formed through a partial region of the grip electrode 36. The power supply terminal 37 may have substantially the same configuration as the power supply terminals 25 and 35 described above with reference to FIG. 2.

FIG. 7 is a schematic block diagram showing control components of a hair care appliance according to an embodiment of the present disclosure.

Referring to FIG. 7, the hair care appliance 1 may include a communication unit 710, an input unit 720, an output unit 730, a current modulator 740, a memory 750, the processor 760, and the power supply 770. The control components shown in FIG. 7 may be an example for convenience of description, and the hair care appliance 1 may include greater or fewer components than the control components shown in FIG. 7.

The communication unit 710 may connect the hair care appliance 1 to an electronic device such as a smartphone, a tablet PC, a PC, or a server. The hair care appliance 1 may transmit and receive data to and from the electronic device through the communication unit 710. For example, the communication unit 710 may include at least one communication module (communication interface) for supporting various wireless communication methods.

The at least one communication module may support a wireless communication method such as WiFi, Bluetooth, or NFC. In some embodiments, when a separate communication terminal is provided in the hair care appliance 1, the at least one communication module may support a wired communication method such as a universal serial bus (USB), a universal asynchronous receiver-transmitter (UART), or RS232.

For example, the at least one communication module may be mounted on the main board 21 of the first care body 2.

The input unit 720 may receive various types of user input related to an operation of the hair care appliance 1. For example, the input unit 720 may include the above-described at least one button 23. The at least one button 23 may receive a physical type of input (push) form the user. The input may include various commands such as power on/off or operation mode setting of the hair care appliance 1. The input unit 720 may also be connected to the main board 21.

The output unit 730 may provide various pieces of information such as an operation state of the hair care appliance 1 to the user. For example, the output unit 730 may include various visual/auditory/tactile output devices such as the speaker 34, the light source 24, a display, or a vibration motor (a haptic module).

The current modulator 740 may adjust the characteristics (a waveform, a size, or a frequency) of the current applied to the care electrodes 22 and 32 through the battery 33.

The memory 750 may store various data including control data for controlling operations of components included in the hair care appliance 1, data received through the communication unit 710, and data for performing an operation based on input acquired through the input unit 720.

The memory 750 may include various storage devices such as ROM, RAM, EEPROM, and flash drive in terms of hardware.

The processor 760 may control the overall operation of the hair care appliance 1. For example, the processor 760 may include at least one micom (microcomputer), integrated circuit, an application specific integrated circuit (ASIC), and an application processor (AP).

The processor 760 may control the current modulator 740 to apply current with predetermined characteristics to the care electrodes 22 and 32 when performing a hair management function.

The current modulator 740, the memory 750, and the processor 760 may be mounted on the main board 21.

The power supply 770 may supply power for operations of components included in the hair care appliance 1. For example, the power supply 770 may include the battery 33 described above with reference to FIG. 2.

When the care electrodes 22 and 32 function as a charging electrode (or a charging terminal), power for charging the battery 33 may be supplied from a cradle (not shown) through the care electrodes 22 and 32 as the hair care appliance 1 is installed on the cradle.

In some embodiments, separate power supply terminals 25 and 35 may be implemented on care bodies, respectively, and in this case, power for charging the battery 33 may also be supplied from the cradle through the power supply terminals 25 and 35.

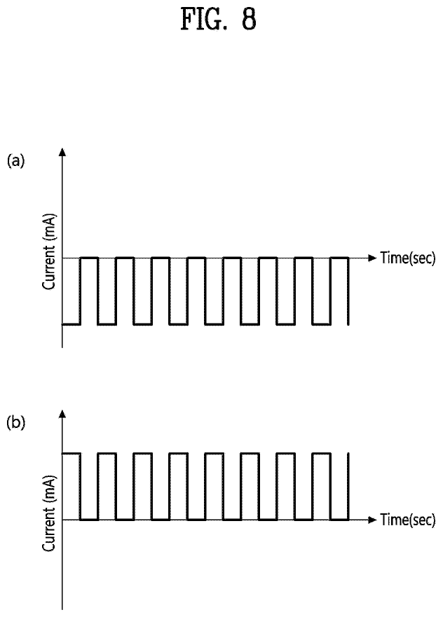

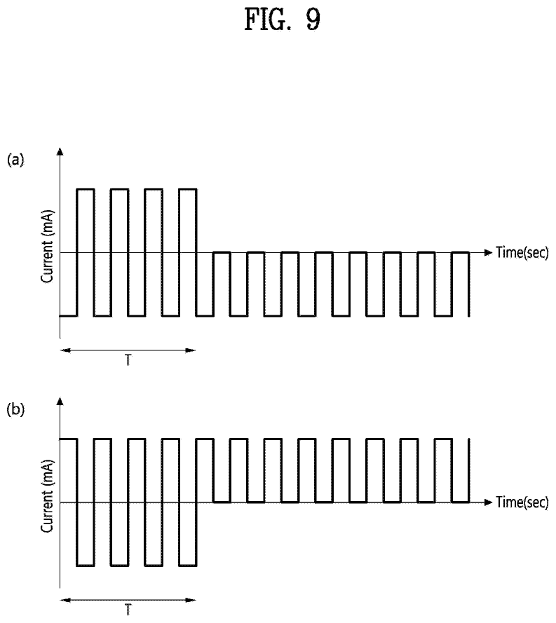

FIGS. 8 to 9 are exemplary graphs related to the characteristics of current applied to care electrodes of a hair care appliance.

When the hair care appliance 1 is implemented according to the embodiment of FIG. 1, current with different polarities may be applied to the first care electrode 22 and the second care electrode 32. For example, current with the characteristics shown in FIG. 8A or 9A may be applied to one of the first care electrode 22 and the second care electrode 32, and current with the characteristics shown in FIG. 8B or 9B may be applied to the other one.

In contrast, when the hair care appliance 1 is implemented according to the embodiment in FIG. 6, current with the same (−) polarity may be applied to the first care electrode 22 and the second care electrode 32. That is, current with the characteristics shown in FIG. 8A or 9A may be applied to each of the first care electrode 22 and the second care electrode 32.

Based thereon, with reference to FIGS. 8 to 9, among the characteristics of the current, the magnitude of current may be related to the strength of electrical attraction or repulsion for penetration into the hair of a nutrient component (active ingredient). That is, as the magnitude of the current increases, the strength of electrical attraction or repulsion may increase, but power consumption may increase instead. Thus, the processor 760 may set the optimal current magnitude for minimizing power consumption while effectively penetrating the nutrient component and may control the current modulator 740.

Among the characteristics of the current, a frequency of the current may be related to an intracellular ion movement speed of the cortex or medulla in the hair. That is, when current of a frequency for accelerating the intracellular ion movement speed is applied, the nutrient component may penetrate into the hair evenly, thereby maximizing a hair care effect. For example, the frequency may be in the range of about 1 KHz to 150 KHz, and in detail, in the range of about 1 KHZ to 50 KHZ.

Comparing FIGS. 8 and 9 in terms of a pattern of current among the characteristics of the current, a current modulator 540 in the embodiment of FIG. 8 may apply current to make each of the care electrodes 22 and 32 continuously have any one polarity.

In contrast, according to the embodiment of FIG. 9, the current modulator 540 may adjust a current pattern to make each of the care electrodes 22 and 32 alternately form a (+) polarity and a (−) polarity for a preset time T (or a predetermined cycle) from a time at which the care electrodes 22 and 32 come in contact with the hair during driving of the hair care appliance 1. For example, for the predetermined time T, a (+) polarity and a (−) polarity may be alternately formed for at least one cycle in the care electrodes 22 and 32.

Like in the embodiment of FIG. 9, when a (+) polarity and a (−) polarity are alternately formed in the care electrodes 22 and 32 for the predetermined time T, activation of movement of substances in cells may be promoted by applying electrical vibration to the inside of hair cells. Thus, movement of the nutrient component penetrating into the hair becomes active, and thus the nutrient component may be effectively provided to various areas in the hair.

That is, the hair care appliance 1 may be effectively driven according to current characteristics for maximizing absorption (penetration) of nutrient components into the hair.

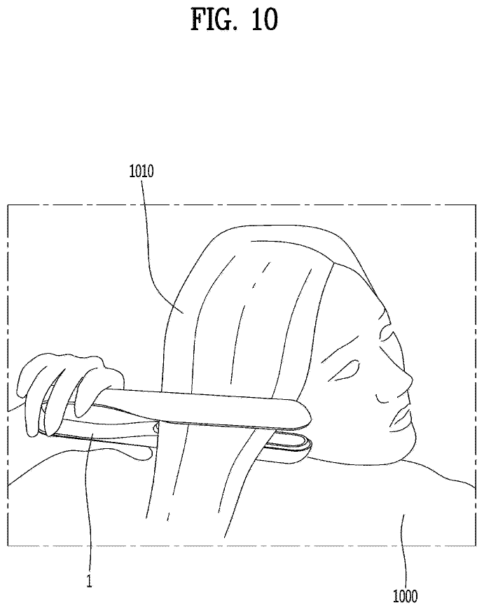

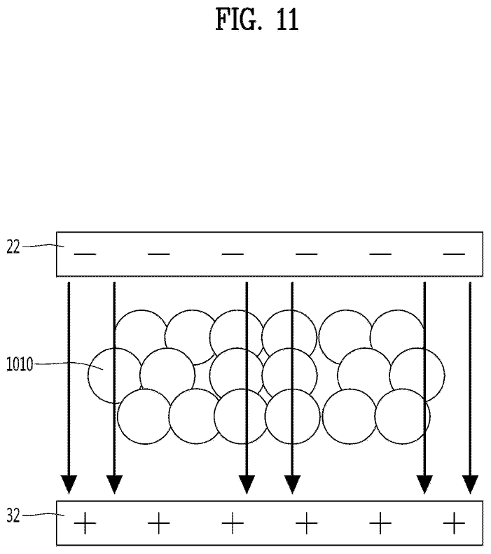

FIG. 10 is a diagram showing the case in which a hair care appliance is used for the hair according to an embodiment of the present disclosure. FIGS. 11 to 13 are diagrams for explaining an operation principle of a hair care appliance according to an embodiment of the present disclosure.

Referring to FIG. 10, a user 1000 may apply nutritional products containing nutrient components (active ingredients) to hair 1010. Then, the user 1000 may hold the hair care appliance 1 with the hand, may position the hair 1010 between the first care body 2 and the second care body 3, and may then press the care bodies 2 and 3 to make the first care electrode 2 and the second care electrode 32 be in close contact with each other.

As the first care electrode 22 and the second care electrode 32 are in close contact with each other, the hair 1010 may be in proximity or contact with the first care electrode 22 and the second care electrode 32.

Referring to FIG. 11, when the hair care appliance 1 is implemented according to the embodiment of FIG. 1, different polarities may be formed in the first care electrode 22 and the second care electrode 32. For example, a (−) polarity may be formed in the first care electrode 22, and a (+) polarity may be formed in the second care electrode 32.

Nutrient components with a (−) polarity present on a surface of the hair 1010 may penetrate into the hair 1010 according to electrical repulsion from the first care electrode 22 and electrical attraction from the second care electrode 32.

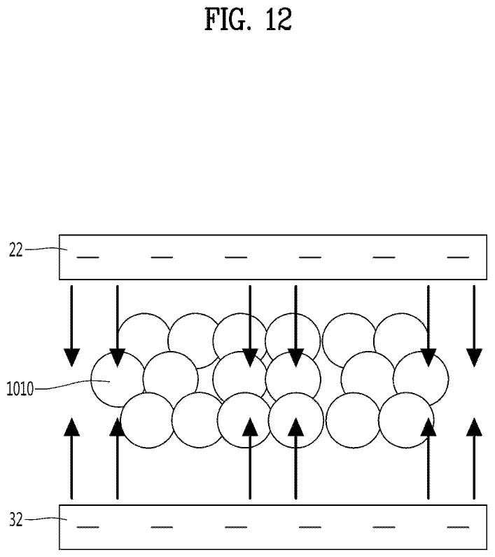

In contrast, referring to FIG. 12, when the hair care appliance 1 is implemented according to the embodiment of FIG. 6, the same (−) polarity may be formed in the first care electrode 22 and the second care electrode 32.

Nutrient components with a (−) polarity present in a surface of the hair 1010 may penetrate into the hair 1010 according to electrical repulsion from each of the first care electrode 22 and the second care electrode 32.

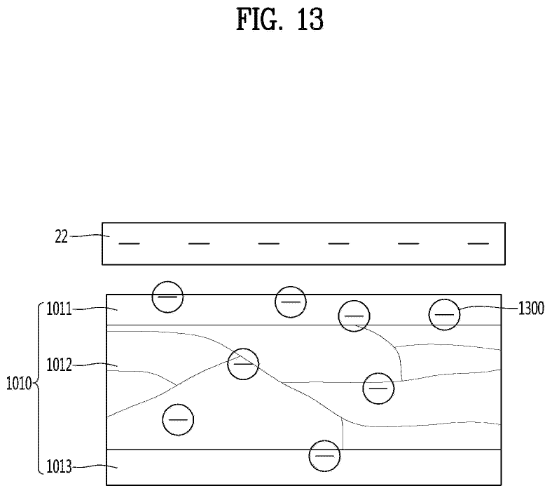

Referring to FIG. 13 with respect to penetration of nutrient components, the hair 1010 may include a cuticle 1011, a cortex 1012 present inside the cuticle 1011, and a medulla 1013 present inside the cortex 1012.

The cuticle 1011 may form the outermost part of the hair and may include keratin hard protein. The cortex 1012 may occupy 85% to 90% of the hair and may include cortical cells and mesenchymal materials. The medulla 1013 may include polygonal cells, which may or may not exist depending on the thickness of the hair.

For example, when the hair 1010 is close to the first care electrode 22, electrical repulsion may be applied from the first care electrode 22 to a nutrient component 1300 present on the surface of the hair 1010.

According to the electrical repulsion, the nutrient component 1300 may penetrate into the cortex 1012 and the medulla 1013 through the cuticle 1011.

That is, when nutritional products including the nutrient component 1300 are simply applied to the hair, most of the nutrient component 1300 may not pass through the cuticle 1011. The hair care appliance 1 according to an embodiment of the present disclosure may improve an absorption rate (permeation rate) of the nutrient component 1300 through an iontophoresis scheme, thereby maximizing a hair care effect.

The above description is merely illustrative of the technical spirit of the present disclosure, and various modifications and variations are possible without departing from the essential characteristics of the present disclosure by those of ordinary skill in the art to which the present disclosure pertains.

Therefore, the embodiments disclosed in the present disclosure are not intended to limit the technical spirit of the present disclosure, but to explain, and the scope of the technical spirit of the present disclosure is not limited by these embodiments.

The scope of the present disclosure needs to be construed by the following claims, and all technical spirit within the equivalent range needs to be construed as being included in the scope of the present disclosure.

1. A hair care appliance comprising:
a first care body having a first care electrode formed thereon;
a second care body having a second care electrode formed thereon; and
a bending part connected between the first care body and the second care body,
wherein the first care body includes:
a main board electrically connected to the first care electrode; and
a processor included in the main board,
wherein the second care body includes a battery electrically connected to the second care electrode, and
wherein the bending part includes:
an elastic housing; and
a flexible printed circuit board (FPCB) accommodated in the elastic housing and electrically connecting the battery and the main board.

2. The hair care appliance of claim 1, wherein the bending part elastically supports the first care body and the second care body to form a predetermined angle.

3. The hair care appliance of claim 2, wherein the first care body and the second care body have a bar shape, respectively,
wherein the first care electrode is formed on a surface of the first care body, which faces the second care body, among surfaces of the first care body, and
wherein the second care electrode is formed on a surface of the second care body, which faces the first care body, among surfaces of the second care body.

4. The hair care appliance of claim 1, wherein the first care electrode is formed adjacent to one end of the first care body, which is not connected to the bending part, among opposite ends of the first care body, and
wherein the second care electrode is formed adjacent to one end of the second care body, which is not connected to the bending part, among of opposite ends of the second care body.

5. The hair care appliance of claim 1, wherein the first care electrode and the second care electrode form different polarities.

6. The hair care appliance of claim 1, further comprising:
a grip electrode formed on one of the first care body and the second care body, and electrically connected to the battery.

7. The hair care appliance of claim 6, wherein the bending part elastically supports the first care body and the second care body to form a predetermined angle, and
wherein the grip electrode is formed on a surface of one of the first care body and the second care body, which does not face another care body, among surfaces of one of the first care body and the second care body.

8. The hair care appliance of claim 6, wherein the grip electrode is formed adjacent to one end of one of the first care body and the second care body, which is connected to the bending part, among opposite ends of the one of the first care body and the second care body.

9. The hair care appliance of claim 6, wherein the first care electrode and the second care electrode form the same polarity, and
wherein the grip electrode forms a different polarity from the first care electrode and the second care electrode.

10. The hair care appliance of claim 9, wherein the first care electrode and the second care electrode form a negative (−) polarity.

11. The hair care appliance of claim 1, wherein the processor performs control to alternately form a positive (+) polarity and a negative (−) polarity in each of the first care electrode and the second care electrode for a preset time from a time at which the first care electrode and the second care electrode come in contact with hair during driving of the hair care appliance.

12. The hair care appliance of claim 1, wherein the processor performs control to alternately form a positive (+) polarity and a negative (−) polarity during at least one cycle in the first care electrode and the second care electrode when the first care electrode and the second care electrode come in contact with hair during driving of the hair care appliance.

13. The hair care appliance of claim 1, wherein the first care body further includes at least one input interface connected to the main board.

14. The hair care appliance of claim 13, wherein the main board is implemented as a double-sided board, and
wherein a first surface of the main board, to which the first care electrode is connected, and a second surface of the main board, to which the at least one input interface is connected, are different from each other.

15. The hair care appliance of claim 1, wherein the elastic housing includes an acrylonitrile butadiene styrene (ABS) or poly oxy methylene (POM) material.

16. The hair care appliance of claim 1, wherein the second care body further includes a sub board that includes a circuit configured to perform a management operation to charge the battery.

17. A hair care appliance comprising:
a first care body having a first care electrode;
a second care body having a second care electrode; and
a bending part connected between the first care body and the second care body,
wherein the first care body includes a main board including a processor,
wherein the second care body includes a battery and a sub board including a battery charging circuit,
wherein the bending part includes a flexible printed circuit board (FPCB) to connect the battery and the main board, and
wherein the first care electrode and the second care electrode respectively form a polarity.

18. The hair care appliance of claim 17, wherein the first care electrode and the second care electrode respectively form a same polarity.

19. The hair care appliance of claim 17, wherein the first care electrode and the second care electrode respectively form different polarities.

20. The hair care appliance of claim 17, wherein the polarity of the first care electrode and the polarity of the second care electrode are respectively used to penetrate a nutrient into a hair of a user.
